(19) United States
(12) Patent Application Publication
(10) Pub. No.: US 2013/0053732 A1
(43) Pub. Date: Feb. 28, 2013

(54) DEVICES AND METHODS FOR TREATING HYPERTENSION WITH ENERGY

(71) Applicant: Richard R. Heuser, Phoenix, AZ (US)

(72) Inventor: Richard R. Heuser, Phoenix, AZ (US)

(21) Appl. No.: 13/217,233

(22) Filed: Aug. 24, 2011

(51) Int. Cl. A61B 18/02; A61N 7/00; A61B 18/18

(52) U.S. Cl. 601/2; 606/33; 606/21

(57) ABSTRACT

Embodiments herein provide systems and methods for the treatment of hypertension. In various embodiments, methods and devices are provided that may be used to treat hypertension in a subject by applying energy, such as radio frequency (RF), microwave, ultrasound, or cryo energy, to one or more renal sympathetic and/or parasympathetic nerves, thereby selectively denervating the renal sympathetic and/or parasympathetic nerves. An exemplary system in accordance with the present disclosure may include a plurality of wires or one or more flexible mesh elements that are extendable from a catheter to expand and contact the inner wall of a conduit at a plurality of points. Additionally, in various embodiments the disclosed methods and devices may be used not only in the renal arteries, but also in the renal veins and/or in the renal pelvis and/or ureter.

### TECHNICAL FIELD

Embodiments herein relate to the field of hemodynamics, and more specifically to devices and methods for the treatment of hypertension.

### BACKGROUND

Hypertension is a cardiac chronic medical condition in which the systemic arterial blood pressure is elevated. Hypertension is classified as either primary (essential) hypertension or secondary hypertension, and about 90-95% of cases are categorized as “primary hypertension,” the causes of which is poorly understood. The remaining 5-10% of cases are classified as secondary hypertension, and are caused by other conditions that affect the kidneys, arteries, heart, or endocrine system.

Persistent hypertension is a risk factor for stroke, myocardial infarction, heart failure and arterial aneurysm, and is a leading cause of chronic kidney failure, and even moderate elevation of arterial blood pressure leads to shortened life expectancy. Dietary and lifestyle changes and medication can improve blood pressure control and decrease the risk of associated health complications in many individuals, however these measures are ineffective or insufficient in some patients with hypertension.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1A and 1B illustrate an example of a device for delivering energy to a renal nerve, the device having a plurality of wires that may be moved between a closed position (FIG. 1A) and an open position (FIG. 1B), in accordance with various embodiments;

FIGS. 2A and 2B illustrate another example of a device for delivering energy to a renal nerve, the device having a plurality of wires that may be coupled together at their distal ends, as illustrated in a close-up view (FIG. 2A) and in a position adjacent a renal nerve (FIG. 2B), in accordance with various embodiments; and

FIGS. 3A and 3B illustrate two examples of a device for delivering energy to a renal nerve via the urinary collecting system and/or renal pelvis, the device having a plurality of fine wires or mesh elements (FIG. 3A) or a single wire or mesh element (FIG. 3B) configured to be moved between a closed position and an open position within the renal pelvis, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide systems and methods for the treatment of hypertension. In various embodiments, methods and devices are provided that may be used to treat hypertension in a subject by applying energy, such as radio frequency (RF), microwave, ultrasound, or cryo energy, to one or more renal sympathetic and/or parasympathetic nerves, thereby selectively denervating the renal sympathetic and/or parasympathetic nerves.

RF energy has been used to treat hypertension previously, but the precise location of the renal sympathetic and parasympathetic nerves is difficult to ascertain, and known methods of denervation can cause damage to surrounding tissues. The renal sympathetic and parasympathetic nerves are adjacent to and wrap generally around (e.g., in casings or layers) the renal artery in humans, and sympathetic denervation has previously been performed using a fairly rigid probe at the tip of a catheter. In such a procedure, an interventionist may perform an arteriogram to determine the location of the renal artery, and then may apply RF energy successively in several sites inside the renal artery (e.g., at 12 o'clock, 3 o'clock, 6 o'clock and 9 o'clock. RF energy may be transmitted through the vessel walls to damage and/or disable the adjacent renal nerves. Examples of this technique are described in “Catheter-Based Renal Denervation for Blood Pressure Reduction,” Krishna Rocha-Singh, Volume: 17 of Cath Lab Digest (available at http://www.cathlabdigest.com/articles/Catheter-Based-Renal-Denervation-Blood-Pressure-Reduction, last checked Jul. 28, 2011), the contents of which are incorporated herein by reference for all purposes.

A problem with this technique is that it requires the interventionist to move the rigid probe (and particularly the energy-emitting tip) around the renal artery multiple times in order to apply energy to multiple locations. Generally, the more the interventionist is required to move around the instrument, the higher the risk of trauma to the vessel wall.

By contrast, in the methods and devices disclosed herein, instead of a rigid probe with a single energy source on the tip, an exemplary apparatus in accordance with the present disclosure may include a plurality of wires or one or more flexible mesh elements that are extendable from a catheter to expand and contact the inner wall of a conduit, such as a vascular or urinary conduit, at a plurality of points. Additionally, in various embodiments the disclosed methods and devices may be used not only in the renal arteries, but also in the renal veins and/or in the renal pelvis. As used herein, the term “conduit” encompasses arteries, veins, and portions of the urinary collection system, such as the renal pelvis and ureters.

In various embodiments, energy (e.g., RF, microwave, ultrasound, or cryo energy) may then be applied to the inner wall of the conduit at each of these contact points, thereby treating the adjacent nerves in multiple locations at once, without requiring the interventionist to move the apparatus around the conduit multiple times. In various embodiments, the device may be deployed in the renal artery, the renal vein, within a ureter, or within a renal capsule.

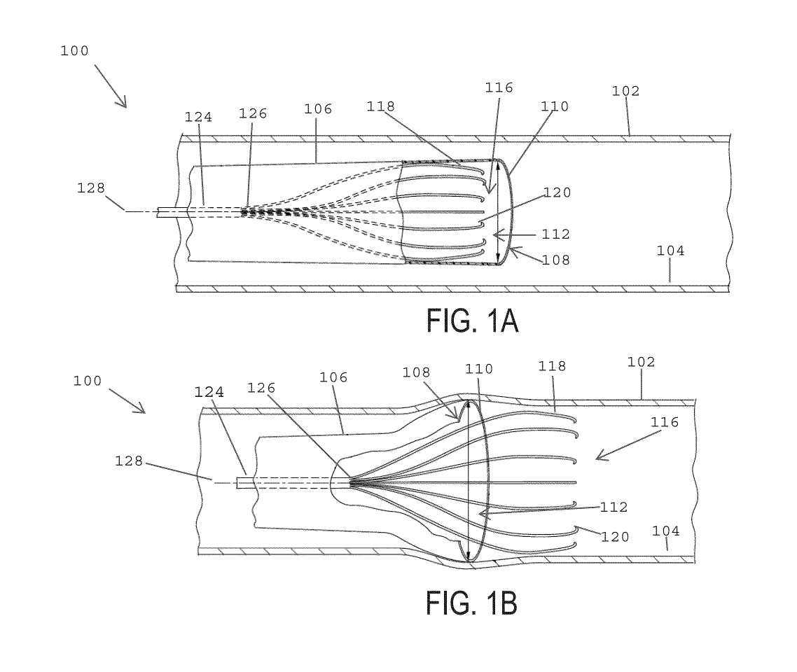

FIGS. 1A and 1B illustrate an example of a device for delivering energy to a renal nerve, the device having a plurality of wires that may be moved between a closed position (FIG. 1A) and an open position (FIG. 1B), in accordance with various embodiments. Referring now to FIGS. 1A and 1B, an exemplary apparatus 100 in accordance with the present disclosure is shown in a vessel 102 with an inner wall 104. In various embodiments, apparatus 100 may include a catheter 106. In various embodiments, catheter 106 may include a distal end 108, an opening 110 adjacent to distal end 108, and a lumen 112 traversing the length of catheter 106 and terminating or being in communication with opening 110. In some embodiments, distal end 108 of catheter 106 may be insertable to a position within vessel 102.

In the embodiment shown in FIGS. 1A and 1B, distal end 108 of catheter 106 may be manipulable between a first configuration where opening 110 has a first diameter labeled ‘A’ in FIG. 1A, and a second configuration where opening 110 has a second diameter labeled ‘B’ in FIG. 1B, although this is not required. In various embodiments, the second diameter B may be greater than the first diameter, as seen in FIGS. 1A and 1B.

In various embodiments apparatus 100 may include a plurality 114 of wires having a plurality 116 of distal ends. Various numbers of wires may be included. For example, in some embodiments, the plurality 114 of wires may include fourteen, sixteen, eighteen, or more wires.

In various embodiments, the plurality 114 of wires may be manipulable between a various positions. For instance, in some embodiments, plurality 114 of wires may be manipulated (e.g., advanced and retracted) between a position wherein the plurality 116 of distal ends are within lumen 112, as shown in FIG. 1A, and another position wherein the plurality 116 of distal ends are extended out of lumen 112 through opening 110, as shown in FIG. 1B. In the embodiment illustrated in FIGS. 1A and 1B, each wire has a main portion 118 and an individual distal end 120. In this illustrated embodiment, distal ends 120 may be separate from one another and not coupled together.

In various embodiments, plurality 114 of wires may be used to deliver sufficient energy to the renal nerve to cause denervation to occur. For example, in embodiments where radiofrequency energy is delivered via plurality 114 of wires, a temperature of about 55-70 degrees Centigrade may be applied for approximately 40 seconds is several (e.g., 3-10) areas of the nerve using approximately 5-10 watts, for example 8 watts, in specific, non-limiting embodiments. In particular embodiments, vascular access may be obtained using conventional means, such as a 6 French system.

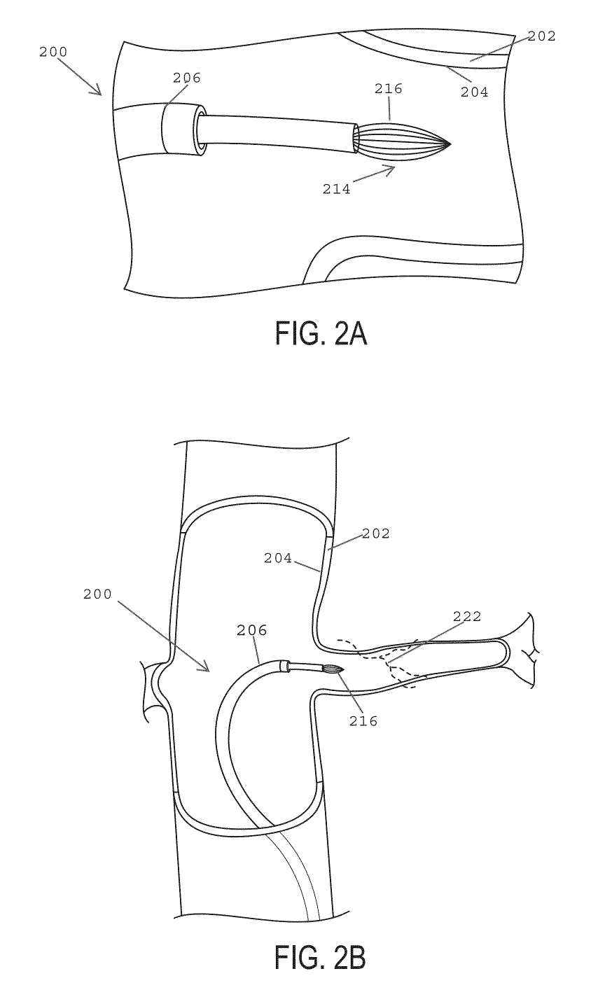

FIGS. 2A and 2B illustrate another example of a device for delivering energy to a renal nerve, the device having a plurality of wires that may be coupled together at their distal ends, as illustrated in a close-up view (FIG. 2A) and in a position adjacent a renal nerve (FIG. 2B), in accordance with various embodiments. Referring now to FIGS. 2A and 2B, the exemplary device 200 is shown in a vessel 202 with an inner wall 204. In various embodiments, apparatus 200 may include an catheter 206. As illustrated, device 200 may be insertable to a position within vessel 202, and may be navigated through one or more bifurcations into renal artery 202a. In the illustrated embodiment, a portion of a renal nerve 222 is shown adjacent renal artery 202a.

In various embodiments, device 200 may include a plurality 214 of wires. Various numbers of wires may be included, for example, fourteen, sixteen, eighteen, or more wires. In the illustrated embodiment, the wires may be coupled together at their distal ends 220. Regardless of whether the distal ends 120, 220 of the wires are formed or coupled together, in some embodiments, the wires may be biased away from one another so that when they are extended from the catheter 106, 206, they expand to contact the inner walls 104, 204 of the vessel 102, 202 at multiple points at once.

The devices disclosed herein, including the examples illustrated in FIGS. 1 and 2, may be used as follows to damage or disable nerves that are adjacent conduits, such as veins, arteries, the renal pelvis, or ureters, in order to treat hypertension. First, with reference to FIGS. 2A and 2B, in various embodiments, the catheter 206 of the device 200 (e.g., which may form a probe of about 0.035 inches in diameter in specific, non-limiting examples) may be positioned adjacent or within a vessel 202 or other conduit, such as one of the renal arteries 202a as illustrated in FIG. 2B. In various embodiments, at lease a portion of renal artery 202a may be adjacent and/or wrapped with renal nerves 222. In various embodiments, once catheter 206 is in a position within vessel 202 adjacent renal nerves 222, wires, such as the plurality 114 of wires illustrated in FIGS. 1A and B or the plurality 214 of wires shown in FIGS. 2A and 2B may be extended out of the distal end of catheter 102, 202. In various embodiments, once extended, because the plurality 114, 214 of wires are biased away from each other, they will expand to contact the inner walls 104, 204 of vessel 102, 202 at as many positions as there are wires.

In various embodiments, energy such as RF (e.g., about 80 degrees centigrade) may then be applied to the conduit wall through each wire (simultaneously or in various sequences) without having to move the wires or the apparatus. In various embodiments, the energy may be transmitted through the conduit wall to the renal nerves 222, thereby damaging or disabling the renal nerves 222.

In various embodiments, the wires such as the plurality 114 of wires shown in FIGS. 1A and 1B, and the plurality 214 of wires shown in FIGS. 2A and 2B may be extended out of the catheter 206 to various lengths. For example, in some embodiments, the plurality 214 of wires may be extended approximately 30 cm from the catheter 206, and may achieve apposition to large expanse of the inner wall 204 of vessel 202. In various embodiments, energy may be applied along the entire length of the extended wires, thereby treating a large portion of vessel 202 (and disabling the adjacent renal nerves 222) in a single operation. In various embodiments, the wires may then be withdrawn back into catheter 206 so that catheter 206 may be removed from the body atraumatically.

In other embodiments, the wires may be extended less distance from catheter 214, about 6 cm for example, so as to avoid unnecessary trauma to the vessel 202 wall if there is not room to extend the wires further. In various embodiments, the wires may be long enough to give the interventionist considerable flexibility in how far the wires are extended out of the catheter 206. In some instances, catheter 206 may be positioned at the orifice of the renal artery 202a coming out of the aorta, so that just the wires extend into the renal artery 202a.

Turning once again to FIGS. 1A and 1B, in some embodiments, each distal end 120 may terminate in an angled portion 124 which may form an angle relative to the rest of the wire. In various embodiments, these angles may be any angle between 0° and 180°, such as 30°, 45°, 90°, or 120°. In various embodiments, each angled portion 124 may be positioned so that it will not contact inner wall 104 of vessel 102, thereby preventing trauma to vessel 102.

In some embodiments, such as those illustrated in FIGS. 1A and 1B, device 100 may include an inner wire 126 which may forms an axis 128 extending through lumen 112. In various embodiments, plurality 114 of wires may include a plurality of proximal ends each seamlessly bound to inner wire 126. In some embodiments, plurality 116 of distal ends may be nominally biased away from axis 128. In some embodiments, such as those shown in FIGS. 1A, 1B, 2A, and 2B, plurality 114 of wires may be curved. In other embodiments, plurality 114 of wires may be straight.

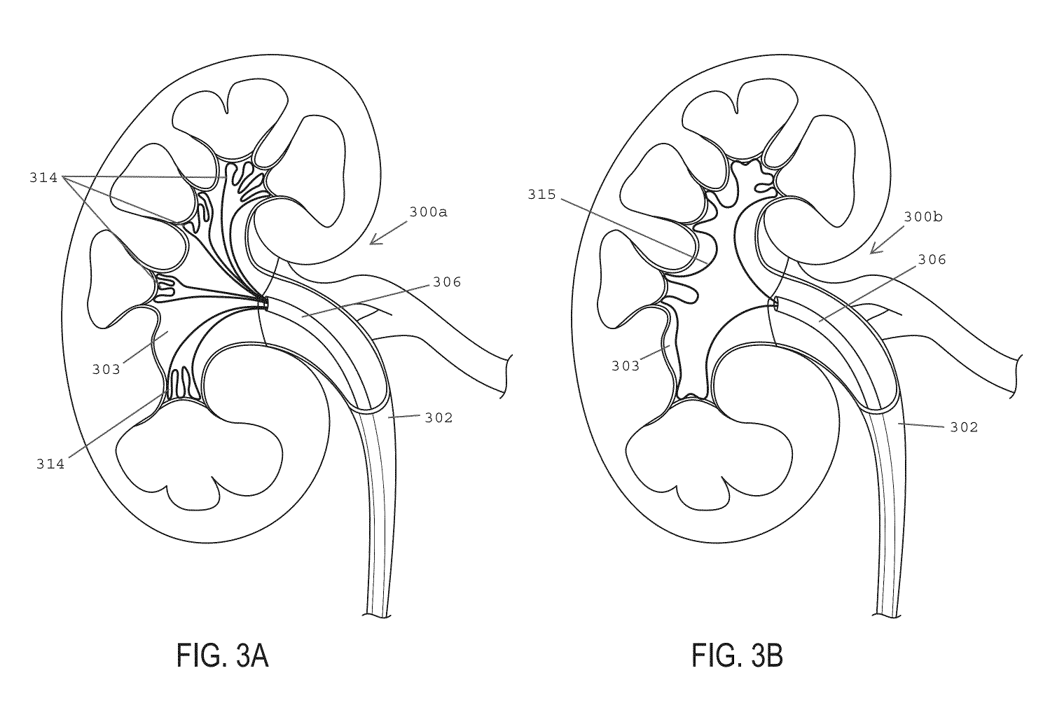

FIGS. 3A and 3B illustrate two examples of a device for delivering energy to a renal nerve via the urinary collection system and/or renal pelvis, the device having a plurality of fine wires or mesh elements (FIG. 3A) or a single wire or mesh element (FIG. 3B) configured to be moved between a closed position and an open position within the renal pelvis, in accordance with various embodiments. Although these embodiments are described and illustrated as being used in the renal pelvis, one of skill in the art will appreciate that the same embodiments also may be used in the renal arteries or renal veins. Referring now to FIGS. 3A and 3B, the exemplary device 300a, 300b is shown with catheter 306 in a portion of the ureter 302 adjacent the renal pelvis 303. As illustrated, device 300a, 300b may be insertable through the urinary collection system (e.g., urethera, bladder, and ureter) to a position adjacent renal pelvis 303.

In various embodiments, device 300a, 300b may include a plurality 314 of wires or fine mesh elements (e.g., as shown in FIG. 3A), or a single wire or fine mesh element 315 (e.g., as shown in FIG. 3B). Various numbers of wires or mesh elements 315 may be included, for example, one, two, three, four, five, six, or more wires or mesh elements.

In various embodiments, the plurality 314 of wires or fine mesh elements or single wire or fine mesh element 315 may be biased into an open position such that it may open (e.g., similar to a fishing net) when catheter 306 is retracted (or when the plurality 314 of wires or fine mesh elements or single wire or fine mesh element 315 is advanced from within the lumen of catheter 306). In various embodiments, the wires or mesh have a high degree of flexibility, and may include a plurality of very compliant struts of wires, for example made from Nitinol™.

In various embodiments, device 300 may be used in the urinary collection system. In various embodiments, device 300 may be passed through the urethra of a male or female subject, into the bladder, and up into the ureter and renal pelvis. In various embodiments, after device 300 is passed from the narrow ureter into the wider renal pelvis, catheter 306 may be withdrawn to expose the plurality 314 of wires or fine mesh elements or single wire or fine mesh element 315. In various embodiments, as the plurality 314 of wires or fine mesh elements or single wire or fine mesh element 315 opens, it may expand to fill all or a portion of the renal pelvis and may generally follow the contours therein.

In some embodiments, device 300 may be used within the upper portion of the ureter or within the renal pelvis, close to the neuroplexis of nerves innervating the renal system. In embodiments, device 300 may be used to apply either radiofrequency energy through a transducer or microwave energy or cryo energy. In particular specific, non-limiting embodiments, a microwave energy may be configured to achieve a temperature in the targeted nerve of about 60 to 75 degrees Centigrade. In other specific, non-limiting embodiments, cryo energy may be applied to lower the temperature of the targeted nerve to a predetermined temperature sufficient to cause denervation in whole or in part. In still other specific, non-limiting embodiments, ultrasound may be applied, either alone or in combination with radiofrequency, microwave, or cryo energy.

In some embodiments, any of devices disclosed herein may be used with commercially available known RF systems such as those manufactured by Olympus, Inc. In various embodiments, the pliability and/or flexibility of the disclosed devices allow use in many areas of the nervous system. This flexibility may be needed in various embodiments, since it is difficult to assess in a living patient where a given set of nerves of interest located. Furthermore, the pliability and/or flexibility of the devices disclosed herein allows precise treatment without damage to delicate vascular and renal tissues.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A device for denervating a renal nerve, the apparatus comprising:
a catheter having a distal end insertable to a first position within a conduit adjacent to the renal nerve, an opening adjacent to the catheter distal end, and a lumen in communication with the opening; and
one or more flexible wires, wherein the one or more flexible wires are manipulable between a first position wherein the one or more flexible wires are within the lumen, and a second position wherein the one or more flexible wires are extended out of the lumen through the opening, the one or more flexible wires being configured to deliver energy to the renal nerve when the one or more flexible wires are in the second position.

2. The device of claim 1, wherein the energy is radiofrequency, microwave, ultrasound or cryo energy.

3. The device of claim 1, wherein the conduit is a renal artery, a renal vein, a ureter, or a renal pelvis.

4. The device of claim 1, wherein the one or more flexible wires comprise a fine mesh or net.

5. The device of claim 1, wherein the one or more flexible wires have a plurality of wire distal ends.

6. The device of claim 5, wherein the one or more wire distal ends are separate from one another.

7. The device of claim 1 wherein the plurality of distal ends each terminates in an angled portion, wherein each angled portion is positioned so that it will not contact an inner wall of the conduit.

8. The device of claim 5, wherein the one or more wire distal ends are coupled together.

9. The device of claim 1, wherein the one or more flexible wires are biased in an open position and configured to expand when extended out of the first lumen in the second position.

10. The device of claim 1, wherein the catheter is an expandable catheter and wherein the catheter distal end is manipulable between a first configuration where the opening has a first diameter and a second configuration where the opening has a second diameter which is greater than the first diameter.

11. The device of claim 1, further comprising an inner wire forming an axis extending through the lumen, wherein the one or more flexible wires further includes one or more proximal ends seamlessly bonded to the inner wire.

12. The device of claim 7, further comprising an inner wire forming an axis extending through the lumen, wherein the one or more flexible wires further includes one or more proximal ends seamlessly bonded to the inner wire, the plurality of distal ends being nominally biased away from the axis.

13. The device of claim 1, wherein the renal nerve is a sympathetic or parasympathetic nerve.

14. A method of denervating a renal nerve, the method comprising:
providing:
a catheter having a distal end insertable to a first position within a conduit adjacent to the renal nerve, an opening adjacent to the catheter distal end, and a lumen in communication with the opening; and
one or more flexible wires, wherein the one or more flexible wires are manipulable between a first position wherein the one or more flexible wires are within the lumen, and a second position wherein the one or more flexible wires are extended out of the lumen through the opening, the one or more flexible wires being configured to deliver energy to the renal nerve when the one or more flexible wires are in the second position;
inserting the catheter to the first position within the conduit, wherein the first distal end is adjacent to the renal nerve;
advancing the one or more flexible wires through the lumen or retracting the catheter so that the one or more flexible wires extend out of the lumen through the opening adjacent the renal nerve; and
delivering an effective amount of energy to the nerve via the one or more flexible wires, thereby denervating the renal nerve.

15. The method of claim 14, wherein the energy is radiofrequency, microwave, ultrasound or cryo energy.

16. The method of claim 14, wherein the conduit is a renal artery, a renal vein, a ureter, or a renal pelvis.

17. The method of claim 14, wherein the one or more flexible wires comprise a fine mesh or net.

18. The method of claim 14, wherein the one or more flexible wires have a plurality of wire distal ends.

19. The method of claim 18, wherein the one or more wire distal ends are separate from one another.

20. The method of claim 14 wherein the plurality of distal ends each terminates in an angled portion, wherein each angled portion is positioned so that it will not contact an inner wall of the conduit.

21. The method of claim 18, wherein the one or more wire distal ends are coupled together.

22. The method of claim 14, wherein the one or more flexible wires are biased in an open position and configured to expand when extended out of the first lumen in the second position.

23. The method of claim 14, wherein the catheter is an expandable catheter and wherein the catheter distal end is manipulable between a first configuration where the opening has a first diameter and a second configuration where the opening has a second diameter which is greater than the first diameter.

24. The method of claim 14, wherein the renal nerve is a sympathetic or parasympathetic nerve.
